(19) United States
(12) Patent Application Publication
(10) Pub. No.: US 2025/0089747 A1
(43) Pub. Date: Mar. 20, 2025

(54) PEA STARCH HMT METHOD

(71) Applicant: ROQUETTE FRERES, Lestrem (FR)

(72) Inventors: Julien PARCQ, LILLE (FR); Jovin HASJIM, BETHUNE (FR); Alban DUPONT, CALONNE SUR LA LYS (FR)

(21) Appl. No.: 18/577,016

(22) PCT Filed: Jul. 5, 2022
(86) PCT No.: PCT/FR2022/051349

§ 371 Date: Jan. 5, 2024

(30) Foreign Application Priority Data
Jul. 8, 2021 (FR) 2107379

(51) Int. Cl. A23L 11/00; A23L 11/30; A23L 29/212; A61K 31/718; C08B 30/06

(52) U.S. Cl. CPC A23L 11/05; A23L 11/31; A23L 29/212; A61K 31/718; C08B 30/06

(57) ABSTRACT

The present invention relates to a method for preparing a legume starch with a high content of slowly digestible fraction (SDS), a heat-moisture treatment method characterized in that it comprises the following steps:
1) Adjusting the water content of the native pea starch to a value of less than 35%, preferably between 20 and 30% by weight, even more preferably between 20 and 25% by weight,
2) Heating the starch thus prepared to a temperature of more than 100° C., preferably from 105° C. to 135° C. for more than 5 hours, preferably for more than 10 hours, and even more preferentially for 24 hours,
3) Recovering and optionally drying the starch thus treated.

The present invention relates to a hydrothermal method for increasing the content of slowly digestible fraction of pea starch. More particularly, this hydrothermal method is a process by heat-moisture treatment, commonly called HMT treatment.

It also relates to the pea starch obtained in this way and uses thereof.

### FIELD OF THE INVENTION

From a physiological perspective, in humans or animals, the bulk of carbohydrates ingested during eating is represented by starch, an energy reserve molecule that is characteristic of plants and a main component of starchy foods (pasta, flour, potatoes).

During digestion, the starch molecules dissociate into smaller glucan chains, themselves dissociated into simple glucose molecules that can be absorbed by the digestive system.

Starch digestion starts in the mouth during mastication by virtue of an enzyme in the saliva: salivary amylase.

This initial breakdown of starch is stopped by the acidity of the stomach but resumes in the duodenum (the first part of the small intestine) by virtue of the action of pancreatic and intestinal enzymes.

The successive action of all of these amylases leads to the appearance of a disaccharide, maltose, which itself is converted into two monosaccharides, glucoses.

Synthesized biochemically, a source of carbohydrates, starch is one of the most widespread organic materials in the plant kingdom, where it constitutes organisms' nutrient reserves.

It is thus naturally present in the reserve organs and tissues of higher plants, in particular in cereal grains (wheat, corn, etc.), legume grains (peas, beans, etc.), tubers (potatoes, yams, etc.), roots (manioc, sweet potato, etc.), bulbs, stems and fruit.

Starch is a primarily mixture of two homopolymers, amylose and amylopectin, composed of D-glucose units bonded to one another via α-(1-4) and α-(1-6) linkages which are the source of branching in the structure of the molecule.

These two homopolymers differ in terms of the degree of branching thereof, and the degree of polymerization thereof.

Amylose is slightly branched with short branches and has a molecular weight between 10,000 and 1,000,000 Dalton. The molecule is formed of 100 to 10,000 glucose molecules.

Amylopectin is a branched molecule with long branches every 24 to 30 glucose units, via α-(1-6) linkages. The molecular weight thereof ranges from 1,000,000 to 100,000,000 Dalton and the degree of branching thereof is the order of 5%. The total chain may include 10,000 to 100,000 glucose units.

The ratio of amylose to amylopectin depends on the botanical source of the starch.

Starch is stored in reserve organs and tissues in a granular state, that is in the form of semi-crystalline granules.

This semi-crystalline state is essentially due to the amylopectin macromolecules.

In the native state, starch granules have a degree of crystallinity which ranges from 15 to 45% by weight which depends substantially on the botanical origin and on the method used for their extraction.

Granular starch placed under polarized light thus has, in microscopy, a characteristic black cross referred to as “Maltese cross”.

This phenomenon of positive birefringence is due to the semi-crystalline organization of the granules: since the average orientation of the polymer chains is radial.

For a more detailed description of granular starch, reference may be made to chapter II, entitled “Structure et morphologie du grain d'amidon” [“Structure and morphology of the starch grain”] by S. Perez, in the work “Initiation à la chimie et à la physico-chimie macromoléculaires” [“Introduction to macromolecule chemistry and physical chemistry”], first edition, 2000, volume 13, pp. 41-86, Groupe Français d'Etudes et d'Applications des Polymères.

Dry starch contains a water content which ranges from 12 to 20% by weight depending on the botanical origin. This water content obviously depends on the residual moisture of the medium (for an a_{w}=1, the starch may fix up to 0.5 g of water per gram of starch).

Heating, with an excess of water, a starch suspension to temperatures close to its gelatinization temperature leads to irreversible swelling of the granules and leads to the dispersion thereof, then the dissolution thereof.

It is these properties in particular which give starch its technological properties of interest.

For a given temperature range, referred to as “gelatinization range”, the starch grain will very quickly swell and lose its semi-crystalline structure (loss of birefringence).

Generally, all the granules will be as swollen as possible over a temperature range of the order of 5 to 20° C. A paste is obtained composed of swollen granules which constitute the dispersed phase, and molecules (mainly amylose) which thicken the aqueous continuous phase.

The rheological properties of the paste depend on the relative proportion of these two phases and on the swelling volume of the granules. The gelatinization range is variable depending on the botanical origin of the starch.

The maximum viscosity is obtained when the starch paste contains a large number of highly swollen granules. When heating is continued with shearing, the granules will burst and the material will disperse in the medium.

Amylose-lipid complexes have delayed swelling because the combination prevents the interaction of the amylose with the water molecules, and temperatures of greater than 90° C. are necessary in order to obtain the total swelling of the granules (amylomaize being complexed to the lipids).

The disappearance of the granules and the dissolution of the macromolecules leads to a reduction in the viscosity.

The lowering of temperature (by cooling) of the starch paste causes gelling or insolubilization of the macromolecules, and crystallization of these macromolecules is observed.

This phenomenon is known by the name retrogradation.

When a paste contains amylose, it is this first molecule which will undergo retrogradation.

It will consist in the formation of a double helix and the combination of the latter to form “crystals” which will give rise to a three-dimensional network via junction zones.

This network is formed very quickly, in a few hours, and continues to develop up to a few weeks later. The association of the molecules with one another via hydrogen bridge bonds forming double helices displaces the associated water molecules in the network and causes significant syneresis.

The structural complexity of the starch and its physico-chemical properties mean that this class of carbohydrate will be assimilated then digested in a variable way in humans and animals.

This is why starch can be classified into three categories, depending on its digestibility: rapidly digestible, slowly digestible, or non-digestible.

Starch, which occurs in naturally granular/semi-crystalline form, can be converted into “rapidly digestible starch” (RDS) after exposure to heat, pressure and/or moisture during food processes.

Slowly digestible starch (SDS) takes longer to be broken down by digestive enzymes compared to RDS because it still has a crystalline structure and is less accessible to digestion enzymes.

Digestion of this SDS fraction leads to a moderate and regular release of glucose into the blood. These are known as low G.I. starches (for “low glycemic index”).

Foods with high SDS content will then elicit lower postprandial glycemic responses and lower postprandial insulin responses than foods with only low SDS content.

Conversely, RDSs are nutritious carbohydrates because they release their glucose into the blood much faster. However, the nutrient source should not contain too much, as this may lead to metabolic syndromes.

As for the so-called resistant starches (RS), these are, in turn, comparable to fibers (such as corn bran, oat fibers, gums) which cannot be digested by intestinal enzymes.

It is accepted in the prior art that total starch is the sum of its three components: RDS, SDS and RS.

The different types of starch are therefore digested at different rates in the human digestive system.

It is therefore assumed that SDS has a slower digestion rate than RDS. RS is a fraction of starch that is resistant to enzymatic digestion in the small intestine. This fraction is fermented in the large intestine and can therefore be considered as dietary fiber.

The SDS and RDS fractions are therefore sources of available glucose.

SDSs are naturally present in some uncooked seeds of cereals such as wheat, rice, barley, rye, corn, and in legumes such as peas, field beans and lentils.

The SDS content is mainly influenced by the gelatinization of starch during the food process which will follow.

Indeed, during this process, exposure to temperature, pressure and moisture leads to the conversion of the SDS fraction into RDS, making the starch more accessible to enzymatic digestion.

This conversion can be minimized by controlling the cooking conditions to limit the gelatinization of the starch.

Therefore, the original content of SDS in the composition or the food product will depend on the way in which its preparation has been carried out.

It is therefore known that food products which contain a lot of SDS are certain pastas, parboiled rice, pearl barley and certain cookies, unlike puffed breakfast cereals or bread which usually contain very little.

The SDS content of foods is conventionally determined using an in vitro method developed by H. N. ENGLYST and his collaborators (published in 1992 in the Eur. J. Clin. Nutr., vol. 46, pp. S33-S50).

In the remainder of this presentation, reference will be made to this 1992 method “according to ENGLYST”.

This method was developed to simulate the enzymatic digestion that occurs in the small intestine.

A sample of product or starch is introduced into a tube, in the presence of digestive enzymes, and the release of glucose is measured during 120 minutes of reaction.

This method makes it possible to differentiate:
The RDS fraction, by measuring rapidly available glucose (RAG); in this case, measuring the glucose released between 0 and 20 minutes;
The SDS fraction, by measuring slowly available glucose (SAG); in this case, measuring the glucose released between 20 and 120 minutes;
The RS fraction, corresponding to glucose not released after 120 minutes, which is calculated according to the Englyst method by the following formula: TS−(RDS+SDS) where TS=Total Starch (considered equal to 100% when the analyses are carried out on the starch as such).

Foods rich in carbohydrates containing more than 50% by weight of available carbohydrates from starch, of which at least 40% by weight are SDS, are conventionally considered to be foods high in SDS.

They are therefore recommended for limiting the glycemic index and insulin production, compared to foods with a lower SDS content.

Of all the starches conventionally used in these food applications, legume starches, and more particularly pea starch, are a prime candidate.

Indeed, pea seeds are known for their high starch content (between 55 and 70% by weight of dry matter) and for their low glycemic index (RATNAYAKE et al. “Pea starch, composition, structure and properties—A review”, Starch/Stärke, 2002, vol. 54, pp. 217-234).

Native pea starches, exhibiting an SDS content conventionally between 27 and 38% by weight according to ENGLYST, are therefore of interest for nutritional applications.

However, in order to prepare foods with high SDS content, it is necessary to use starch with a higher fraction of slowly digestible carbohydrate.

It is known in the prior art that annealing-type hydrothermal treatments or other HMT hydrothermal treatments make it possible to alter the crystal structure of the starch granule.

Annealing is a term used in polymer science to describe the optimization of the crystallization by heating a polymer to a temperature below its melting point, in order to obtain the growth of crystalline areas, perfection of crystals and a change to more stable crystal structures.

During the annealing process, it is assumed that the starch granules undergo limited but reversible swelling without destroying the granular and molecular structure or the solubilization of the molecules of the starch polymers.

However, it is known in the prior art that annealing processes do not have the primary objective of increasing the level of the slowly digestible fraction (SDS), but rather of making the starch more digestible, and in particular legume starch such as pea (cf. article by CHUNG et al., in Carbohydr. Polym., 2009, vol. 75, pp. 436-447), by increasing the RDS fraction.

In its patent application WO 2021/099747, the Applicant company, however, selected to optimize this annealing technology, not to increase the RDS fraction, but rather to increase the SDS content of legume starch, especially pea starch, by seeking and finding annealing process conditions that are particularly suited to this purpose.

The HMT treatment is a heat treatment carried out on starch with limited moisture (<35%) at a high temperature (>100° C.) for a certain time.

Due to the limited moisture, the gelatinization temperature of the starch is higher than in an excess of water (Donovan, Biopolymers, 1979, vol. 18 pp. 263-275), and the starch can therefore be heated to a temperature above 100° C. without causing gelatinization.

During the HMT treatment, the molecules in the starch granules have increased local mobility.

Therefore, the crystalline structure of the starch granules is modified, which changes its gelatinization and digestibility properties.

The applicant company has therefore chosen to experiment with this technology to propose an alternative treatment to that taught by its patent application WO 2021/099747.

It has thus found operating conditions that make it possible to increase the level of slowly digestible starch (SDS) in the native pea starch, more particularly to convert the resistant starch (RS) fraction of the native pea starch into slowly digestible starch (SDS), by modifying the crystalline structure of the native pea starch by HMT.

### BRIEF DESCRIPTION OF THE DRAWINGS

Other features, details and advantages will appear from reading the following detailed description, and by analyzing the appended drawings in which:

FIG. 1 shows the thermogram of sample 1C.

FIG. 2 shows the thermogram of sample 1B.

### DETAILED DESCRIPTION

Thus, the invention relates to a method for preparing a legume starch, preferably pea starch, with a high content of slowly digestible fraction (SDS), a heat-moisture treatment (HMT) method characterized in that it comprises the following steps:
1) Adjusting the water content of the native pea starch to a value of less than 35%, preferably between 20 and 30% by weight, even more preferably between 20 and 25% by weight,
2) Heating the starch thus prepared to a temperature of more than 100° C., preferably from 105° C. to 135° C. for more than 5 hours, preferably for more than 10 hours, and even more preferentially for 24 hours,
3) Recovering and optionally drying the starch thus treated.

In the meaning of the present invention, “high content of slowly digestible fraction” is understood to mean an SDS content increase of 5 to 25% by dry weight, preferably 10 to 20% by dry weight with respect to the starch from which it is prepared.

For the purposes of the present invention, “legume” means any plant belonging to the families of the cesalpiniaceae, mimosaceae or papilionaceae, and particularly any plant belonging to the family of the papilionaceae, for example pea, bean, broad bean, field bean, lentil, alfalfa, clover or lupin.

This definition includes in particular all the plants described in the tables contained in the article by HOOVER et al. entitled “Composition, structure, functionality and chemical modification of legume starches: a review”, Can. J. Physiol. Pharmacol. 1991, vol. 69, pp. 79-92).

Preferably, the legume is selected from the group comprising pea, bean, broad bean and field bean.

Advantageously, it is pea, the term “pea” being considered here in its broadest sense and including in particular:
all the wild-type varieties of “smooth pea”, and
all the mutant varieties of “smooth pea” and of “wrinkled pea”, regardless of the uses for which said varieties are usually intended (human food, animal feed and/or other uses).

Said mutant varieties are in particular those called “mutants r”, “mutants rb”, “mutants rug 3”, “mutants rug 4”, “mutants rug 5” and “mutants lam” as described in the article by C-L HEYDLEY et al., entitled “Developing novel pea starches”, Proceedings of the Symposium of the Industrial Biochemistry and Biotechnology Group of the Biochemical Society, 1996, pp. 77-87.

According to another advantageous variant, legumes (for example varieties of pea or field bean) are plants giving grains containing at least 25%, preferably at least 40%, by weight of starch (dry/dry).

“Legume starch” is intended to mean any composition extracted, by any means, from a legume and in particular from a papilionaceae, the starch content of which is greater than 40%, preferably greater than 50% and even more preferentially greater than 75%, these percentages being expressed as dry weight relative to the dry weight of said composition.

Advantageously, this starch content is greater than 90% (dry/dry). It may in particular be greater than 95% by weight, including greater than 98% by weight.

“Native” starch means a starch which has not undergone any chemical modification.

In order to determine their base content of SDS fraction, pea starches, according to the invention or not, are analyzed according to the in vitro digestion process conditions of the method by ENGLYST et al in “Classification and measurement of nutritionally important starch fractions”, Eur. J. Clin. Nutr., 1992, vol. 46 (Supp. 2), pp. S33-S50.

The method consists of measuring the fractions of rapidly digestible starch (RDS), slowly digestible starch (SDS) and non-digestible (resistant) starch (RS) contained in a food.

These fractions are determined after enzymatic digestion with pancreatin, amyloglucosidase and invertase.

The released glucose is measured by colorimetry, using a Glucose GOD FS glucose oxidase kit, referenced 1 2500 99 10 923, marketed by the company DiaSys Distribution France Sarl, following the protocol of said kit.

The detail of the method implemented for measuring the digestion according to ENGLYST is similar to that given by the applicant company in its patent application WO 2021/099747.

### Reagents Used:

Anhydrous sodium acetate (ref: 71184, from SIGMA)
Benzoic acid (ref: 242381, from SIGMA)
CaCl₂) (ref: 1.02378.0500, from MERCK)
Acetic acid 0.1M (ref: 33209, from SIGMA)
Porcine Pancreatin 8×USP (ref: P 7545 from SIGMA)
Amyloglucosidase EC 3.2.1.3 (from SIGMA, with activity ≥260 U/mL/≈300 AGU/mL, Cat. NO. A7095)
Invertase EC 3.2.1.26 (from SIMA, with activity ≥300 units/mg solid, Cat. NO. I-4504)
Guar (ref: G4129, from SIGMA)
Ethanol at 66°

### Procedure

The acetate buffer (0.1 M) was prepared by dissolving 8.203 g of anhydrous sodium acetate in 250 ml of saturated benzoic acid solution, with the diluent to 500 mL with RO water, by adjusting the pH to 5.2 with 0.1 M acetic acid, by diluting it again to 1000 mL with RO water and adding 4 mL of CaCl₂) 1 M per liter of buffer.

The enzymatic solution was prepared freshly before the experiments. Four 50 mL centrifuge tubes were prepared, each containing 2.5 g of porcine pancreatin (8×USP, P7545, Sigma) and mixed with 20 mL of RO water. The mixture was stirred for 10 minutes and centrifuged for 10 minutes at 1500×g.

The supernatants (13.5 mL of each tube) were combined and mixed with 2.775 mL of amyloglucosidase (EC 3.2.1.3, A7095, Sigma), 3.225 mL of RO water and 33.3 mg of invertase (EC 3.2.1.26, I4504, Sigma) pre-dissolved in 4 mL of RO water.

Each sample (0.8 g, dry base) was mixed with 20 mL of acetate buffer and 50 mg of guar gum in a 50 ml tube.

A “blank” control was prepared using 20 ml of acetate buffer and 50 mg of guar gum, without sample, while a standard contained 0.5 g of anhydrous glucose and 50 mg of guar gum in 20 mL of acetate buffer solution.

Guar gum can be pre-dissolved in the acetate buffer, for example, 750 mg of guar gum in 300 mL of acetate buffer.

The samples, the blank and the standard were equilibrated at 37° C. in a water bath with stirring for 15 minutes.

An aliquot (0.1 mL) was taken into each tube before adding the enzymes (0 minute) and mixed with 0.9 mL of 66% ethanol solution.

By taking one tube per minute, 5 mL of enzymatic solution were added to the samples, to the blank and to the standard.

Immediately after mixing, the tubes were placed in the water bath at 37° C. for 120 min under stirring.

An aliquot (0.1 mL) was taken from each tube at 20 and 120 minutes and mixed with 0.9 mL of 66% v/v ethanol solution.

The mixtures of alcohol solutions were centrifuged at 1500×g for three minutes.

The glucose content (G₀, G₂₀ and G₁₂₀ for 0, 20 and 120 minutes, respectively) in each supernatant was analyzed using a colorimetric method, and used to calculate the rapidly digestible starch (RDS), slowly digestible starch (SDS) and resistant starch (RS) as follows:

$$RDS = ( {G}_{20} - {G}_{0} ) \times 0.9 \\ SDS = ( {G}_{120} - {G}_{20} ) \times 0.9 \\ RS = 100 \% - ( RDS + SDS ) = 100 \% - ( {G}_{120} \times 0.9 )$$

The conventional ENGLYST method does not make it possible to hydrolyze the starch samples until exhaustion, since, as the applicant company has observed it, a larger amount of starch can be hydrolyzed after 2 hours of reaction.

This observation made it possible for the applicant company to exploit this property by revealing the existence of a very slowly digestible fraction, resulting from the RS fraction of pea starch, in its patent application WO 2021/099748. This fraction was defined as the vSDS fraction (for very Slowly Digestible Starch).

Therefore, the AOAC 2002.02 method, which uses 16-hour hydrolysis, was used to obtain the absolute RS content, and the result may be claimed as a dietary fiber.

To differentiate between the two levels of RS, the RS_{E} and RS_{A} parameters were used to denote the RS levels obtained by the ENGLYST method (RS_{E}) and by AOAC 2002.02 (RS_{A}), respectively.

The difference between RS_{E} and RS_{A} is considered to be very slowly digestible starch (vSDS), the digestible part of starch which requires more than 2 hours to be hydrolyzed using the ENGLYST method.

According to this method, the native pea starch conventionally has a content:
of RDS between 13 and 16% by weight,
of SDS between 24 and 38% by weight,
of RS_{E} between 50 and 65% by weight,
of RS_{A} between 9 and 20% by weight,
of vSDS between 35 and 45% by weight,
   These values are given with a standard deviation of +/−2%, given the intrinsic variability during the ENGLYST enzymatic test.

To increase the SDS level, the heat-moisture treatment (HMT) method according to the invention, developed by the Applicant company, uses a precise hydrothermal approach.

The invention thus relates to a method for preparing a legume starch, preferably pea starch, with a high content of slowly digestible fraction (SDS), a hydrothermal treatment method characterized in that it comprises the following steps:
1) Adjusting the water content of the native pea starch to a value of less than 35%, preferably between 20 and 30% by weight, even more preferably between 20 and 25% by weight,
2) Heating the starch thus prepared to a temperature of more than 100° C., preferably from 105° C. to 135° C. for more than 5 hours, preferably for more than 10 hours, and even more preferentially for 24 hours,
3) Recovering and optionally drying the starch thus treated.

The first step of said method according to the invention consists of adjusting the water content of the native pea starch to a value of less than 35%, preferably between 20 and 30% by weight, even more preferably between 20 and 25% by weight,

The second step of the method according to the invention consists of heating the starch thus prepared to a temperature of more than 100° C., preferably from 105° C. to 135° C. for more than 5 hours, preferably for more than 10 hours, and even more preferentially for 24 hours,

Depending on the water content of the native pea starch chosen, the applicant company recommends adapting the treatment temperature.

In a first preferred embodiment of the method according to the invention, at a water content of the order of 25%, as will be exemplified below, it is preferred to heat, for 24 hours, to a temperature on the order of 105° C., while at a reduced water content, on the order of 20%, it is preferred to heat, for 24 hours, at a higher temperature, on the order of 135° C. These two treatments will lead to significantly increasing the SDS content (from 10 to 20%) without increasing the RDS content of more than 5 to 10%).

In a second preferred embodiment of the method according to the invention, at a water content of the order of 30% and a temperature of the order of 105° C., or a water content of the order of 25% and a temperature of the order of 120° C., the increase in the SDS content (from 4 to 15%) is accompanied by an increase in the RDS content of 10 to 30%. These combinations of moisture and temperature content actually lead to a higher degree of gelatinization of the pea starch, leading to a greater digestibility of carbohydrate structures of native pea starch than in the first embodiment of the method of the invention.

In a third embodiment of the method according to the invention, at a water content of the order of 20% and a temperature of 105° C. or 120° C., these intermediate treatment conditions can make it possible to increase the SDS content, even if less amplitude.

The third and final step of the method in accordance with the invention thus consists of recovering and potentially drying the starch milk treated in this way, as exemplified hereinafter.

The residual moisture content of the obtained dry starch is less than 15% by weight, preferably less than or equal to 12% by weight.

The Englyst digestibility measurement of these products gives SDS values increased by 5 to 25% by dry weight, preferably 10 to 20% by dry weight with respect to the initial starch.

As will be shown below, this SDS value for pea starch is above 35% by dry weight, preferably between 40 and 50% by dry weight.

The present invention also relates to a pea starch with a high content of slowly digestible fraction prepared according to one of the methods described above, characterized in that the SDS content is greater than 35% by weight, preferably between 40 and 50% by weight.

These starches with high SDS content will then be advantageously used in fields of application relating to food (intended especially for sportspersons) or medicine (specialist nutrition).

The invention also relates to the use of a starch according to the invention in food and medical fields of application, especially for food for sportspersons or specialist nutrition.

The invention will be better understood on reading the following examples, which are intended to be illustrative, only mentioning certain embodiments and certain advantageous properties according to the invention, and are non-limiting.

### Example 1: Treatment of a First Batch of Native Pea Starch Having an SDS Content on the Order of 33%

The water content of a pea starch batch (LN 30 pea starch sold by the applicant company) was adjusted to 20, 25 or 30% by stirring in a food processor (Thermomix TM3300, Vorwerk, Germany).

Samples whose moisture was adjusted were sealed in glass jars and left at equilibrium overnight at room temperature.

The samples in the sealed pots were then heated in an oven at 105, 120 and 135° C. for 24 hours. The details of the treatments are indicated in Table I.

If necessary, the HMT samples were dried using a fluidized bed dryer (TG200, Retch) at 60° C. to a moisture equal to or less than 12%, and ground using the Thermomix TM3300.

TABLE I

| Reference of the | Water content after | Temperature | Duration of |
|---|---|---|---|
| treated batches | precisely (%) | (° C.) | treatment (h) |
| 1A | 20 | 105 | 24 |
| 1B | 25 | 105 | 24 |
| 1C | 30 | 105 | 24 |
| 2A | 20 | 120 | 24 |
| 2B | 25 | 120 | 24 |
| 3A | 20 | 135 | 24 |

The in vitro digestibility of the treated pea starch was analyzed according to ENGLYST as indicated above, and the results presented in Table II below.

TABLE II

| | RDS (%) | SDS (%) | RS_{E} (%) | RS_{A} (%) | vSDS (%) |
|---|---|---|---|---|---|
| Pea starch | 16 | 33 | 51 | 10 | 41 |
| LN 30 | | | | | |
| 1A | 20 | 30 | 50 | 7 | 43 |
| 1B | 18 | 44 | 38 | 8 | 30 |
| 1C | 46 | 37 | 17 | 7 | 10 |
| 2A | 19 | 26 | 55 | 8 | 47 |
| 2B | 37 | 41 | 22 | 8 | 14 |
| 3A | 25 | 48 | 27 | 7 | 20 |

Except for treatments 1A and 2A, all the HMT samples showed a lower RS_{E} content and a higher SDS content than the native counterpart.

All the HMT samples also showed a higher RDS content than the native counterpart. The results indicate that the HMT weakened the crystalline structure of the native pea starch and increased its digestibility, but did so while maintaining the low-to-moderate RDS content.

The highest SDS content originated from the sample of the treatment 3A, followed by that of the treatment 1B.

The treatments 1C and 2B produced the highest RDS since these treatments involve high moisture and/or high temperature.

As described above, the starch's gelatinization temperature decreases with increasing moisture content.

Therefore, the processing samples 1C and 2B could have the highest degree of gelatinization, as indicated their enthalpy of gelatinization lower than that of the other samples and the native pea starch (Table 3 below).

The RS_{A} content of the native pea starch has slightly decreased after the HMT. However, there were large differences between the RS_{E} and RS_{A}, which indicates that most RS_{E} in native pea starch and the samples treated could be hydrolyzed after 2 hours of digestion, that is to say, very slowly digestible starch (vSDS).

The gelatinization properties were analyzed using DSC 8000 (Perkin Elmer, USA).

Each starch sample was mixed with water to obtain a suspension of starch at 18% (w/w).

The starch suspension (15 mg) was placed in an aluminum crucible and hermetically sealed. It was then equilibrated at 5° C. before being heated from 5° C. to 110° C. at 10° C./min. The onset temperature (Tₒ), the peak temperature (Tₚ), the conclusion temperature (T_{c}) and the enthalpy of gelatinization was determined from their thermograms.

The results obtained are shown in Table III below.

TABLE III

| | Tₒ | Tₚ₁ | Tₚ₂ | T_{c} | ΔH |
|---|---|---|---|---|---|
| | (° C.) | (° C.) | (° C.) | (° C.) | (J/g of starch) |
| LN 30 | 63.4 | 71.2 | ND* | 78.2 | 13.6 |
| pea starch | | | | | |
| 1A | 67.7 | 75.3 | ND | 81.2 | 13.7 |
| 1B | 70.7 | Shoulder | 84.2 | 88.5 | 13.6 |
| 1C | 78.8 | Shoulder | 88.5 | 92.2 | 13.4 |
| 2A | 70.7 | 74.4 | 83.5 | 88.5 | 13.9 |
| 2B | 67.7 | 77.1 | 88.0 | 91.7 | 12.3 |
| 3A | 67.7 | 75.9 | 86.3 | 90.3 | 13.7 |

*Not detected

An increase in the gelatinization temperature (Tₒ, Tₚ and T_{c}) was observed for all HMT samples, indicating that the crystal structure has been modified by the treatments.

With the exception of treatment 1A, the other HMT samples showed an additional Tₚ at >80° C. (Tₚ2).

For treatments 1B and 1C, the Tₚ2 was more dominant than the Tₚ1, where the latter appeared as a shoulder.

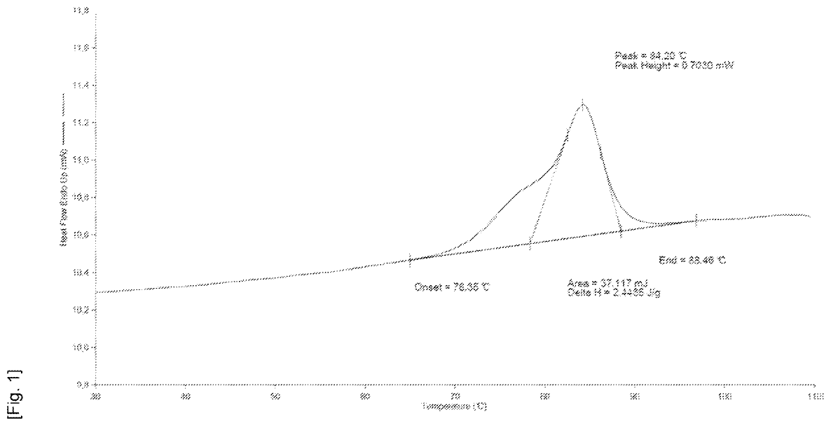

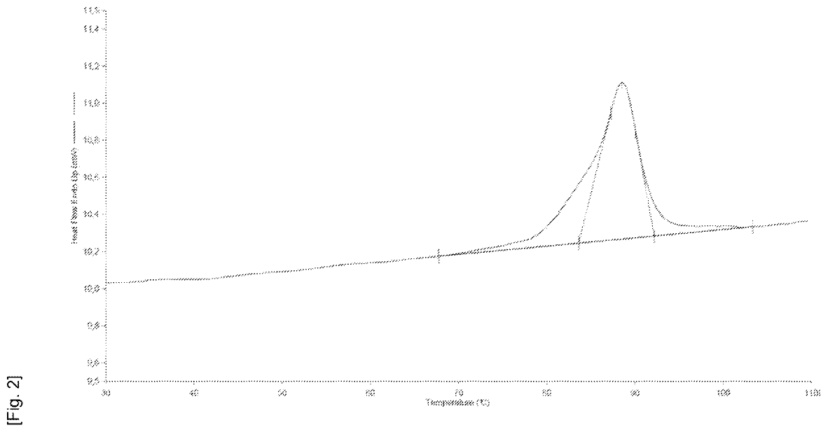

In fact, the shoulder of the sample after treatment 1C was smaller than that after treatment 1B (see FIGS. 1 and 2), probably due to the higher moisture in the first.

The results also indicated that the HMT samples were more heat-resistant than the native pea starch.

### Example 2: Treatment of a Second Batch of Native Pea Starch Having an SDS Content on the Order of 24%

The water content of a batch of native pea starch (native pea starch N-735 sold by the applicant company) was adjusted as in example 1.

TABLE IV

| Reference of the | Water content after | Temperature | Duration of |
|---|---|---|---|
| treated batches | precisely (%) | (° C.) | treatment (h) |
| 4A | 20 | 105 | 24 |
| 4B | 25 | 105 | 24 |
| 4C | 30 | 105 | 24 |
| 5A | 20 | 120 | 24 |
| 5B | 25 | 120 | 24 |
| 6A | 20 | 135 | 24 |

The samples were tested for their in vitro digestibility and their gelatinization properties, following the same methods as in example 1.

TABLE V

| | RDS (%) | SDS (%) | RS_{E} (%) | RS_{A} (%) | vSDS (%) |
|---|---|---|---|---|---|
| Native pea | 15 | 24 | 61 | 17 | 44 |
| starch N-735 | | | | | |
| 4A | 15 | 34 | 51 | 10 | 41 |
| 4B | 18 | 43 | 39 | 8 | 31 |
| 4C | 42 | 37 | 21 | 6 | 15 |
| 5A | 14 | 36 | 50 | 9 | 40 |
| 5B | 35 | 39 | 26 | 6 | 20 |
| 6A | 21 | 41 | 38 | 8 | 30 |

All HMT samples also showed a higher SDS content and RS_{E} content lower than the native counterpart, indicating that the HMT has weakened the crystalline structure of the native pea starch and has increased its digestibility.

Except for treatments 4C and 5B, the RDS content levels remained low (21% or less). As in example 1, the two highest SDS content levels originated from the samples of treatments 4B and 6A.

As in example 1, treatments 4C and 5B produced the highest RDS because these treatments involve a high moisture and/or temperature. Consequently, these samples could have the highest degree of gelatinization, as indicated by their gelatinization enthalpy lower than that of the other samples and of the native pea starch (Table VI below).

The RS_{A} content of the native pea starch has slightly decreased after the HMT. However, as in example 1, there were large differences between the RS_{E} and RS_{A}, indicating most RS_{E} in the native pea starch and the samples treated were vSDS.

The gelatinization properties are determined as in example 1.

TABLE VI

| | Tₒ | Tₚ₁ | Tₚ₂ | T_{c} | ΔH |
|---|---|---|---|---|---|
| | (° C.) | (° C.) | (° C.) | (° C.) | (J/g starch) |
| Native pea | 61.0 | 69.3 | ND | 78.2 | 13.6 |
| starch N-735 | | | | | |
| 4A | 67.4 | 75.9 | ND | 82.5 | 14.2 |
| 4B | 69.7 | Shoulder | 82.2 | 88.0 | 12.7 |
| 4C | 66.2 | Shoulder | 85.0 | 89.5 | 11.9 |
| 5A | 70.6 | Shoulder | 81.6 | 87.5 | 13.1 |
| 5B | 66.9 | 76.5 | 86.9 | 91.0 | 11.5 |
| 6A | 67.6 | 75.9 | 86.3 | 90.3 | 12.3 |

As in example 1, an increase in the gelatinization temperature (Tₒ, Tₚ and T_{c}) was observed for all HMT samples, indicating that the crystal structure has been modified by the treatments. With the exception of treatment 4A, the other HMT samples showed a Tₚ>80° C.

It seems that there were two populations of granules after the HMT treatment, possibly because the limited moisture was not uniformly distributed among the starch granules.

The results also indicate that the HMT samples were more heat-resistant than the native pea starch.

1. A method for preparing a legume starch with a high content of slowly digestible fraction (SDS), a heat-moisture treatment (HMT) method wherein it comprises the following steps:
1) adjusting the water content of the native pea starch to a value of less than 35%, preferably between 20 and 30% by weight, even more preferably between 20 and 25% by weight,
2) heating the starch thus prepared to a temperature of more than 100° C., preferably from 105° C. to 135° C. for more than 5 hours, preferably for more than 10 hours, and even more preferentially for 24 hours,
3) recovering and optionally drying the starch thus treated.

2. The method according to claim 1, wherein the legume starch is selected from the group of pea, bean, broad bean, field bean, lentil, alfalfa, clover and lupine starches, and is particularly pea starch.

3. The method according to claim 1, wherein the high content of slowly digestible fraction (SDS) corresponds to an increase of 5 to 25% by weight, preferably 10 to 20% by dry weight with respect to the initial starch.

4. The method according to claim 1, wherein it consists of adjusting the water content of the native pea starch at 25% by weight and heating it to 105° C. or of adjusting its water content to 20% by weight and heating it to 135° C., so as to increase the SDS content by 10 to 20% relative to the initial native pea starch without increasing the RDS content by more than 5 to 10%.

5. The method according to claim 1, wherein it consists of adjusting the water content of the native pea starch at 30% by weight and heating it to 105° C. or of adjusting its water content to 25% by weight and heating it to 120° C., so as to increase the SDS content by 4 to 15% relative to the initial native pea starch without increasing the RDS content by more than 10 to 30% relative to the initial native pea starch.

6. A pea starch with a high content of slowly digestible fraction prepared according to the method of claim 1, wherein the SDS content is greater than 35% by weight, preferably between 40 and 50% by weight.

7. Use of the starch according to claim 6 in food and medical fields of application, especially for food for sportspersons or specialist nutrition.
